(19) United States
(12) Patent Application Publication
(10) Pub. No.: US 2025/0169502 A1
(43) Pub. Date: May 29, 2025

(54) Plant Growth Promoting Bradyrhizobium Compositions

(71) Applicant: PROTEALIS NV, Ghent (BE)

(72) Inventors: Serge BEULLENS, Leuven (BE); Jan MICHIELS, Leuven (BE); Sofie GOORMACHTIG, Ghent (BE); Judith VAN DINGENEN, Ghent (BE); Robbe BOMBEKE, Ghent (BE); Juan Sebastian RAMIREZ PRADO, Ghent (BE)

(21) Appl. No.: 18/839,719

(22) PCT Filed: Feb. 23, 2023
(86) PCT No.: PCT/EP2023/054535

§ 371 Date: Aug. 19, 2024

(30) Foreign Application Priority Data
Feb. 24, 2022 (EP) 22158375.0

(51) Int. Cl. A01N 63/20; A01P 21/00

(52) U.S. Cl. CPC A01N 63/20; A01P 21/00

(57) ABSTRACT

The present invention relates to the field of sustainable agriculture. In particular, the present invention relates to an isolated Bradyrhizobium bacterium having enhanced characteristics. including but not limited to enhanced adaptation to the low and medium root zone temperature for example in North-West Europe.

### FIELD OF THE INVENTION

The present invention relates to the field of sustainable agriculture. Specifically, the invention provides microbial compositions and methods for improving the yield of nitrogen fixing plants. In particular, the present invention relates to an isolated Bradyrhizobium bacterium having enhanced characteristics, including but not limited to enhanced adaptation to low and/or medium soil temperatures, for example in North-West Europe.

### BACKGROUND

Soybean (Glycine max) is one of the most important legume crops worldwide producing protein-rich beans (about 40% of the seed) with all essential amino acids for human food and animal feed (Hammond et al 2003 Beans, Oxford Academic Press). The cultivation of soybean in North-West Europe is challenging because the plant is not adapted to the cold and wet environment. In the last decade, extensive breeding programs and field trials with hundreds of early-maturing soybean cultivars have been performed to find genotypes suitable for growth in more temperate regions such as Belgium (Aper et al 2015 Plant Genetic Resources 1; Pannecoucque et al 2018 J Agri Sc 156:3) and Germany (Zimmer et al 2016 Eur J Agron 72). As a result, dozens of soybean cultivars (such as Primus, Bettina and Shouna cv.) have been bred to be adapted to local environmental conditions in terms of tolerance to common fungal pathogens in combination with high seed yields and protein content. However, when grown in reference soils, none of these cultivars meet the bean protein threshold of >42% that has been set by the industry to be eligible for processing for human consumption (Pannecoucque et al 201J Agri Sci 156:3). To guarantee high protein contents in beans, soybean plants need to engage in a symbiotic interaction with bacteria from nitrogen-fixing genera, the so-called rhizobia. This symbiosis results in the formation of root nodules in which the bacterial strains fix atmospheric nitrogen that is subsequently used for plant protein production. Soybean plants can interact with diverse bacterial genera such as Rhizobium, Bradyrhizobium, Ensifer (Sinorhizobium), Azorhizobium, but also (Para)Bulkholderia (Gyaneshwar et al 2011 MPMI 24; Ramirez et al 2019 Microbes Environ 34; Sharaf et al 2019 Microbiome 7). Currently, soybean cultivation in North-West Europe uses commercially available Bradyrhizobium inoculants leading to variable, unsatisfactory results (Pannecoucque et al 2018 J Agri Sc 156:4; Zimmer et al 2016 Eur J Agron 72). These non-endemic bacterial strains are possibly not adapted to the Northern environmental conditions resulting in insufficient nodulation and subsequently inadequate bean protein contents for the processing industry (Alexandre and Oliveira 2013 Crit Rev Microbiol 39; Pannecoucque et al 2018 J Agri Sc 156:3). Especially the low root zone temperature is a major constraint in the establishment of the legume-rhizobia symbiosis (Schmidt et al 2015 Plant Soil 397; Zhang et al 1995 Environ Exp Bot 35). Optimal temperatures for soybean growth and nodule formation are between 25 and 30° C. which is significantly higher than the North-West Europe soil temperatures that ranges from 8 and 15° C. at the sowing time (Miransari et al 2013 J Plant Growth Regul 32). Hence, there is a need to identify new plant symbiotic bacteria that are adapted to low root zone temperatures and that promote yield of leguminous crops both quantitatively and/or qualitatively.

### SUMMARY

A trapping experiment was set up in diverse Belgian soils to capture native soybean nodulators. One of these nitrogen fixing bacteria is disclosed herein and significantly outperforms the most closely related B. japonicum strains in terms of growth at lower temperatures. Therefore, the invention provides in a first aspect, an isolated Bradyrhizobium japonicum strain having the deposit accession number LMG P-32018, or having at least 99.90% genomic sequence identity with the strain having the deposit accession number LMG P-32018, wherein said isolated strain has retained the plant growth and/or yield characteristics at temperatures between 10 and 25° C. of said deposited strain LMG P-32018. Also an enriched culture and a biologically pure culture of said Bradyrhizobium strain are provided. In another aspect, a composition comprising said Bradyrhizobium strain or one of said cultures is provided. In one embodiment, the Bradyrhizobium strain is lyophilized, freeze-dried, dried in a form of a powder or present as an aqueous slurry. In another embodiment, said composition further comprises growth medium appropriate for Bradyrhizobium japonicum species and/or a cryoprotectant. The composition may also further comprise an agriculturally compatible carrier. In another aspect a plant seed coated with the Bradyrhizobium strain of the invention is provided. In one embodiment, the plant seed is a leguminous plant seed, more particularly a soybean.

Given that the Bradyrhizobium strain disclosed in current application is especially useful in the field of agriculture, methods are provided for enhancing growth, yield and/or nitrogen fixation of plants, more particularly of leguminous plants by administration of said Bradyrhizobium to said plants. This is equivalent as saying that the use of the Bradyrhizobium strain of the invention, the cultures or compositions comprising it is provided to enhance growth, yield and/or nitrogen fixation of a plant, more particularly a leguminous plant. In one embodiment, said yield refers to the protein content of the seeds, more particularly of soybeans. It is envisaged that the Bradyrhizobium strain of the invention can be administered by several ways, for example but not limited to by coating plant seeds, inoculating the soil or other plant growth supporting media, spraying or irrigating plants. Hence, in one embodiment, the methods comprise the steps of inoculating a plant growth medium with the Bradyrhizobium strain according to the invention or with one of the cultures or compositions comprising it, and growing the leguminous plant in said plant growth medium. In a particular embodiment, the Bradyrhizobium strain, cultures or compositions are applied to the plant growth medium as a powder, as a pellet, as a granule or as a liquid. In other embodiments, the methods comprise the steps of growing plants in an environment that supports plant growth, and administering a sprayable formulation to said environment or to said plant, said formulation comprising the Bradyrhizobium strain of the invention or the cultures or compositions comprising it.

### Deposit of Biological Material

Purified cultures of the microbial strain described in present application were deposited by VIB vzw (Rijvisschestraat 120, 9052 Gent, Belgium) at the BCCM (Belgian Coordinated Collections of Microorganisms) consortium (BCCM represented by Laboratorium voor Microbiologie—Bacterienverzameling (LMG), Universiteit Gent, K. L. Ledeganckstraat 35, 9000 Gent, Belgium), recognized as an International Depositary Authority by the World Intellectual Property organization since Mar. 1, 1992 and in accordance with the Budapest Treaty as specified in Rule 31(1) EPC2000 for the purpose of patent procedure and the regulations thereunder. The Bradyrhizobium japonicum strain of current application has been deposited as Bradyrhizobium japonicum RHG_Soy_223 with deposit number LMG P-32018. The original deposit has been done on 05.10.2020.

### Figure Legends

FIG. 1 shows the average number of nodules per plant of soybean cultivar Shouna (white) and Primus (gray). Plants (n=3) were inoculated with 0.01 OD of Bradyrhizobium japonicum strain G49, strain 532C or strain Soy223. After 4 weeks nodule presence/number was assessed.

FIG. 2 are exemplary illustrations of root nodules induced by B. japonicum strain Soy223 on soy cultivars Primus (left) and Shouna (right).

FIG. 3 shows the chlorophyl content (SPAD values) measured on three soybean cultivars (S19L0511, S19L0512 and Shouna) inoculated with a mock control, B. japonicum strain RHG_Soy_223 (soy_223) or strain USDA6 (USDA_6).

FIG. 4 illustrates the growth of the B. japonicum strain RHG_Soy_223 (SOY223) and strain USDA6 at 20° C. based on Bioscreen data. The OD of strain RHG_Soy_223 (green) and USDA6 (orange) was determined during 8 days and 12 hours. The graph shows mean and error of 5 repeats with SEM plotted.

FIG. 5 illustrates the growth of the RHG_Soy_223 (SOY223) and USDA6 strain at 30° C. based on Bioscreen data. The OD of strain RHG_Soy_223 (green) and USDA6 (orange) was determined during 8 days. The graph shows mean and error of 5 repeats with SEM plotted.

FIG. 6 shows the average count of CFU/seed on soy seeds coated with a commercial seed sticker and either comprising B. japonicum strain of RHG_Soy_223 or G49, over a time span of 60 days starting from the day of seed coating.

FIG. 7 shows the average survival rate of B. japonicum strains on soy seeds coated with a commercial seed sticker and either comprising B. japonicum strain of RHG_Soy_223 or G49, over a time span of 60 days starting from the day of seed coating.

FIGS. 8 and 9 show the normalized growth of various B. japonicum strains (RHG_Soy_223, G49, USDA6, and 532C) in YMB media when incubated at either 20° C. (FIG. 8) or 30° C. (FIG. 9) measured at OD600.

FIGS. 10 and 11 show the chlorophyll content (SPAD values—FIG. 10) and number of nodules (FIG. 11) of soy plants (Artemis) grown in cold conditions (22° C. day/10° C. night) from seeds inoculated with either B. japonicum strains USDA6, G49, or RHG_Soy_223.

### DETAILED DESCRIPTION

The massive soybean import by Europe for food and feed production causes a negative socio-economic and environmental impact. Hence, there is an increasing interest in the local production of soy. Various breeding efforts already resulted in varieties that are able to grow in European colder climates. However, to ensure high protein rich beans, it is essential that soy establishes a symbiosis with nitrogen (N₂)-fixing bacteria in their root nodules. Currently, the use of commercial inoculants in Nord-West European soy agriculture has shown to be variable and insufficient for high quality harvests. In order to get efficient nodulation as soon as possible after sowing and thus early in the growing season, it is imperative to find bacterial strains that are adapted to the medium and cold soil temperatures in North-West Europe and that initiate nodulation of soybean roots at the start of the growing season when the temperature of the soils is still low. To solve this problem, the inventors of current application initiated a nodulation trap experiment during which three early-maturing soybean cultivars were grown in natural conditions in over 100 different garden soils. In the majority of these soils, root nodules were found. Genetic analysis revealed a large bacterial richness and diversity amongst Rhizobia and Bradyrhizobia strains present in these different nodules.

In current application a novel nitrogen fixing bacterial strain is disclosed, more particularly a Bradyrhizobium japonicum strain, even more particularly the B. japonicum strain RHG_Soy_223. The strain has high genetic homology to B. japonicum strain USDA6 but compared to the publicly available strains, RHG_Soy_223 shows improved growth characteristics, e.g. improved growth at medium and at high temperature conditions. The strain of the application is able to undergo a nitrogen fixing interaction with a leguminous plant, more particularly with soy. “Nitrogen-fixing interaction” as used herein refers to a plant-bacterial interaction in which atmospheric nitrogen is made available to the plant as a nutrient through the bacterium. Typically, this interaction results in the formation of root nodules containing the bacterium.

### Strain

In one aspect of current application, a Bradyrhizobium strain is provided comprising a 16S rRNA sequence as depicted in SEQ ID No. 1. In one embodiment, said Bradyrhizobium strain is a nitrogen fixing bacterial strain. In another embodiment, said Bradyrhizobium strain is a Bradyrhizobium japonicum strain, more particularly the Bradyrhizobium japonicum RHG_Soy_223 strain. In another embodiment, said strain is an isolated strain. The term “isolated” means that the bacterial strain has been removed from its natural environment. “Isolated” thus implies a purification step. However, “isolated” does not necessarily reflect the extent to which the microorganism, more particularly the bacterium has been purified. A bacterial strain of current application is purified at least 2×, at least 5×, at least 10×, at least 50× or at least 100× from the raw material from which it is isolated. As a non-limiting example, if a microorganism is isolated from soil as raw material, the microorganism can be isolated to an extent that its concentration in a given quantity of purified or partially purified material (e.g. soil) is at least 2×, at least 5×, at least 10×, at least 50× or at least 100× that in the original raw material.

In yet another embodiment, said B. japonicum strain shows a statistically significantly improved growth compared to other publicly available B. japonicum strains, more particularly to strain USDA6. In one embodiment, said improved growth is measured at a temperature between 10° C. and 25° C., or between 20° C. and 30° C., or between 15° C. and 22° C. In yet another embodiment, said B. japonicum strain improves the nitrogen status and/or nitrogen fixation of a soybean plant that was inoculated with said strain compared to other publicly available B. japonicum strains, more particularly to strain USDA6. With “nitrogen status” as used herein it is meant the nitrogen level or nitrogen concentration or nitrogen usage in the inoculated plant.

(16S rRNA of Bradyrhizobium japonicum RHG_Soy_223)
SEQ ID No. 1
CAAACTCCTACGGGAGGCAGCAGTGGGGAATATTGGACAATGGGGGCAAC
CCTGATCCAGCCATGCCGCGTGAGTGATGAAGGCCCTAGGGTTGTAAAGC
TCTTTTGTGCGGGAAGATAATGACGGTACCGCAAGAATAAGCCCCGGCTA
ACTTCGTGCCAGCAGCCGCGGTAATACGAAGGGGGCTAGCGTTGCTCGGA
ATCACTGGGCGTAAAGGGTGCGTAGGCGGGTCTTTAAGTCAGGGGTGAAA
TCCTGGAGCTCAACTCCAGAACTGCCTTTGATACTGAGGATCTTGAGTTC
GGGAGAGGTGAGTGGAACTGCGAGTGTAGAGGTGAAATTCGTAGATATTC
GCAAGAACACCAGTGGCGAAGGCGGCTCACTGGCCCGATACTGACGCTGA
GGCACGAAAGCGTGGGGAGCAAACAGGATTAGATACCCTGGTAGTCCACG
CCGTAAACGATGAATGCCAGCCGTTAGTGGGTTTACTCACTAGTGGCGCA
GCTAACGCTTTAAGCATTCCGCCTGGGGAGTACGGTCGCAAGATTAAAAC
TCAAAGGAATTGACGGGGGCCCGCACAAGCGGTGGAGCATGTGGTTTAAT
TCGACGCAACGCGCAGAACCTTACCAGCCCTTGACATGTCCAGGACCGGT
CGCAGAGATGTGACCTTCTCTTCGGAGCCTGGAGCACAGGTGCTGCATGG
CTGTCGTCAGCTCGTGTCGTGAGATGTTGGGTTAAGTCCCGCAACGAGCG
CAACCCCCGTCCTTAGTTGCTACCATTTAGTTGAGCACTCTAAGGAGACT
GCCGGTGATAAGCCGCGAGGAAGGTGGGGATGACGTCAAGTCCTCATGGC
CCTTACGGGCTGGGCTACACACGTGCTACAATGGCGGTGACAATGGGATG
CTAAGGGGCGACCCTTCGCAAATCTCAAAAAGCCGTCTCAGTTCGGATTG
GGCTCTGCAACTCGAGCCCATGAAGTTGGAATCGCTAGTAATCGTGGATC
AGCACGCCACGGTGAATACGTTCCCGGGCCTTGTACACACCGCCCGTCAC
ACCATGGGAGTTGGTTTT

In another aspect of current application a bacterial strain is provided with deposit accession number LMG P-32018. In one embodiment, said bacterial strain is a nitrogen fixing bacterial strain. In another embodiment, said bacterial strain is a Bradyrhizobium sp. strain, more particularly a Bradyrhizobium japonicum strain. In a most particular embodiment, said bacterial strain is the Bradyrhizobium japonicum RHG_Soy_223 strain with deposit number LMG P-32018. In another embodiment, said strain is an isolated strain.

In an aspect of the current application, an isolated Bradyrhizobium japonicum strain is provided having the deposit accession number LMG P-32018, or having at least 99.90%, preferably at least 99.91%, preferably at least 99.92%, preferably at least 99.93%, preferably at least 99.94%, preferably at least 99.95%, preferably at least 99.96%, preferably at least 99.97%, preferably at least 99.98%, more preferably at least 99.99% genomic sequence identity with the strain having the deposit accession number LMG P-32018, wherein said isolated strain has retained the plant growth and/or yield characteristics at temperatures between 10 and 25° C. of said deposited strain LMG P-32018.

In an embodiment, said strain has at least one 16S rRNA according to SEQ ID No. 1. In another embodiment, said strain has at least one 16S rRNA comprising one or more synonymous mutations in its 16S rRNA sequence compared to SEQ ID No. 1.

A “synonymous mutation” refers to a change in the DNA/RNA sequence that codes for amino acids in a protein sequence, but does not change the encoded amino acid. Due to the redundancy of the genetic code (multiple codons code for the same amino acid), these changes usually, but not only, occur in the third position of a codon.

In an embodiment, said strain shows improved growth at 20 to 30° C. when grown in yeast mannitol broth (YMB), compared to Bradyrhizobium japonicum strain USDA6, strain G49, and/or strain 532C.

In an embodiment said strain is capable of inducing nitrogen-fixing nodules, preferably red nitrogen-fixing nodules on roots of a plant. Preferably said plant is a leguminous plant, preferably of the genus Glycine, such as Glycine max.

In an embodiment said strain is capable of inducing more nitrogen-fixing nodules on roots of a plant compared to Bradyrhizobium japonicum strain USDA6, or strain G49. Preferably said strain is capable of inducing at least 5% more, preferably at least 6% more, preferably at least 7% more, preferably at least 8% more, preferably at least 9% more, preferably at least 10% more nitrogen-fixing nodules. Preferably said plant is a leguminous plant, preferably of the genus Glycine, such as Glycine max.

In another or further embodiment, said strain is capable of inducing more nitrogen-fixing nodules on roots of a plant compared to Bradyrhizobium japonicum strain USDA6, or strain G49. Preferably said strain is capable of inducing at least 5% more, preferably at least 6% more, preferably at least 7% more, preferably at least 8% more, preferably at least 9% more, preferably at least 10% more nitrogen-fixing nodules when grown in colder conditions (22° C. day/10° C. night). Preferably said plant is a leguminous plant, preferably of the genus Glycine, such as Glycine max.

In an embodiment, said strain increases the nitrogen status in a plant when inoculated with said strain as measured via increased Soil Plant Analysis Development (SPAD) values, compared to said plant inoculated with Bradyrhizobium japonicum strain USDA6 or strain G49. Preferably said plant is a leguminous plant, preferably of the genus Glycine, such as Glycine max.

In another or further embodiment, said strain increases the nitrogen status in a plant when inoculated with said strain as measured via increased Soil Plant Analysis Development (SPAD) values, compared to said plant inoculated with Bradyrhizobium japonicum strain USDA6 or strain G49 when grown in colder conditions (22° C. day/10° C. night). Preferably said plant is a leguminous plant, preferably of the genus Glycine, such as Glycine max.

The “Soil Plant Analysis Development” or “SPAD” numerical SPAD value specifies the relative content of chlorophyll within the sample leaf. It can be measured using a hand-held device that is used for accurate, quick, and non-destructive in situ measurements of chlorophyll concentrations for numerous plant species. The SPAD chlorophyll meter is one of the most commonly used diagnostic tools to measure crop nitrogen status.

In an embodiment said strain increases the leaf chlorophyll content in a plant inoculated with said strain compared to a seed of said plant inoculated with Bradyrhizobium japonicum strain USDA6. Preferably said plant is a leguminous plant, preferably of the genus Glycine, such as Glycine max.

In an embodiment said strain increases the protein content in a seed of a plant inoculated with said strain. Preferably said plant is a leguminous plant, preferably of the genus Glycine, such as Glycine max.

From here on, any of the above bacterial strains or Bradyrhizobium strains or Bradyrhizobium japonicum RHG_Soy_223 strain with deposit number LMG P-32018 will be referred to as the bacterial strain of current application. Also RHG_Soy_223 is interchangeably used with Soy_223, RHG_Soy223 or Soy223.

In another aspect of the application a culture of the bacterial strain of current application is provided. The term “culture” as used herein refers to a population of microorganisms that are propagated on or in media of various kinds. In one embodiment, said culture is an enriched culture of the bacterial strain of current application. This is equivalent as saying that a culture of microorganisms, more particularly a bacterial culture, is provided, wherein said culture is enriched with the bacterial strain of current application (i.e. Bradyrhizobium japonicum RHG_Soy_223) and wherein “enriched” means that the total microbial (or more particularly the total bacterial) population of said culture contains more than 50%, more than 60%, more than 70%, more than 80%, more than 90%, or more than 95% of the isolated bacterial strain of current application, more particularly of Bradyrhizobium japonicum RHG_Soy_223 with deposit number LMG P-32018.

In another embodiment, a biologically pure culture of the bacterial strain of current application is provided. As used herein, “biologically pure” refers to a culture which contains substantially no other microorganisms than the desired strain and thus a culture wherein virtually all of the cells present are of the selected strain. In practice, a culture is defined biologically pure if the culture contains at least more than 96%, at least more than 97%, at least more than 98% or at least more than 99% of the bacterial strain of current application, more particularly of Bradyrhizobium japonicum RHG_Soy_223 with deposit number LMG P-32018. When a biologically pure culture contains 100% of the desired microorganism a monoculture is reached. A monoculture thus only contains cells of the selected strain and is the most extreme form of a biologically pure culture.

In yet another embodiment, the culture of the Bradyrhizobium strain of the application comprises at least 1%, at least 5%, at least 10%, at least 25%, at least 50% or at least 75% living Bradyrhizobium japonicum RHG_Soy_223 bacteria. In a particular embodiment, the bacterial strain of current application may be lyophilized, freeze-dried or in a form of a dry powder. In another particular embodiment, an aqueous slurry of the bacterial strain of the current application or of any culture herein described comprising the strain is provided, the slurry being optionally dried to a powder at a temperature which does not adversely affect viability of the bacterial strain.

Compositions

In another aspect, a composition is provided comprising the bacterial strain of current application. This is equivalent as saying that the composition comprises an inoculum of the bacterial strain of the application. As used herein, the term “inoculum” is intended to mean any form of bacterial cells, or spores, which is capable of propagating on or in the soil when the conditions of temperature, moisture, etc., are favourable for bacterial growth. A “spore” generally refers to a microorganism in its dormant, protected state.

In a particular embodiment, the composition may be in the form of a liquid, a slurry, a wettable powder or a dry powder. In a particular embodiment, the bacterial strain of current application may be lyophilized, freeze-dried or in the form of a dry powder before it is used in the processing of the composition. In another particular embodiment, an aqueous slurry of the bacterial strain of the current application is provided, which is optionally dried to a powder at a temperature which does not adversely affect viability of the bacterial strain. The powder may then be mixed with an agriculturally compatible carrier. In other embodiments, a liquid suspension or slurry of the bacterial strain of the current application may be applied to an absorbent material, e.g. a granular mass, or may be used to coat plant seeds or other plant tissues. Also a powder comprising the bacterial strain of the application is suitable for coating seeds. When used to coat plant seeds, the composition may be applied to the seeds and allowed to dry. In embodiments wherein the composition is a powder (e.g. a wettable powder), a liquid, such as water, may need to be added to the powder before application to a seed.

In another embodiment, a composition is provided comprising the bacterial strain of current application further comprising a cryoprotectant and/or growth medium appropriate for Bradyrhizobium japonicum species. A “cryoprotectant” as used herein protects the bacteria by preventing the damaging effects of water crystals when cells are frozen, more particularly at −60° C., or −70° C. or −80° C. or in liquid nitrogen. Non-limiting examples of a cryoprotectant is glycerol and trehalose. In another embodiment, a composition is provided comprising the bacterial strain herein disclosed wherein the bacterial strain is lyophilized, freeze dried or in the form of a dry powder. In one embodiment, the composition can further comprise a preservative.

In another particular embodiment, any of the compositions described herein further comprises an agriculturally compatible carrier. Said carrier can be inert (e.g. a detectable agent or label or liquid carrier) or active (e.g. a fertilizer), but should allow the bacterial strain of the application to remain efficacious and viable. An “agriculturally compatible carrier” may be a natural or synthetic, organic or inorganic material with which the active compounds (e.g. the bacterial strain of the current application) are combined to facilitate their application on the plant, a plant part, plant seed or to the plant growth medium. Said “agriculturally compatible carrier” which can be regarded as a vehicle, is generally inert and it must be acceptable in agriculture. Thus, the phrase “agriculturally compatible” denotes a substance that can be used routinely under field conditions without interfering with growers' planting equipment, and without adversely influencing crop development or the desired ecological balance in a cultivated area.

The agriculturally compatible carrier can be solid. Solid carriers can include but are not limited to clays, natural or synthetic silicates, silica, resins, waxes, solid fertilizers, a polymer, a granular mass, perlite, a perlite granule, peat, a peat pellet, soil, vermiculite, charcoal, sugar factory carbonation press mud, rice husk, carboxymethyl cellulose, fine sand, calcium carbonate, flour, alum, a starch, talc, polyvinyl pyrrolidone, or a combination thereof. The agriculturally compatible carrier can be a liquid. In one embodiment, the liquid carrier is water, sugar water, diluted or non-dilute growth medium to culture the bacterial strain of the application. Non-limiting examples of suitable growth media for said bacterial strain include yeast extract mannitol (YEM), yeast mannitol agar (YMA), yeast mannitol broth (YMB). Other non-limited example of liquid carriers can include but are not limited to water, sugar wateralcohols, ketones, petroleum fractions, oils, aromatic or paraffinic hydrocarbons, chlorinated hydrocarbons, liquefied gases or a combination thereof. More particularly, the agriculturally compatible carrier can include a dispersant, a surfactant, an additive, a thickener, an anti-caking agent, residue breakdown, a composting formulation, a granular application, diatomaceous earth, a colouring agent, a stabilizer, a preservative, a polymer, a coating or a combination thereof.

The carrier can also be a slurry, optionally comprising a sticking agent capable of sticking the inoculum to the substrate of interest, for example to a plant seed. Non-limiting examples of sticking agents include alginate, mineral oil, syrup, gum arabic, honey, methyl cellulose, milk, wallpaper paste, and combinations thereof. One of the ordinary skills in the art can readily determine the appropriate carrier to be used taking into consideration factors such as a particular bacterial strain, plant to which the inoculum is to be applied, type of soil, climate conditions, whether the inoculum is in liquid, solid or powder form, and the like. The additive can comprise an oil, a gum, a resin, a clay, a polyoxyethylene glycol, a terpene, a viscid organic, a fatty acid ester, a sulfated alcohol, an alkyl sulfonate, a petroleum sulfonate, an alcohol sulfate, a sodium alkyl butane diamate, a polyester of sodium thiobutant dioate, a benzene acetonitrile derivative, a proteinaceous material, or a combination thereof. The proteinaceous material can include a milk product, wheat flour, soybean meal, blood, albumin, gelatin, or a combination thereof. The thickener can comprise a long chain alkylsulfonate of polyethylene glycol, polyoxyethylene oleate or a combination thereof. The surfactant can contain a heavy petroleum oil, a heavy petroleum distillate, a polyol fatty acid ester, a polyethoxylated fatty acid ester, an aryl alkyl polyoxyethylene glycol, an alkyl amine acetate, an alkyl aryl sulfonate, a polyhydric alcolhol, an alkyl phosphate, or a combination thereof. The anti-caking agent can include a sodium salt such as a sodium sulfite, a sodium sulfate, a sodium salt of monomethyl naphthalene sulfonate, or a combination thereof, or a calcium salt such as calcium carbonate, diatomaceous earth, or a combination thereof. The agriculturally compatible carrier can also include a fertilizer, a micronutrient fertilizer material, an insecticide, a herbicide, a plant growth amendment, a fungicide, a molluscicide, an algicide, a bacterial inoculant, a fungal inoculant, or a combination thereof. Non-limiting examples are provided below. As way of example the bacterial strain of the current application may be mixed with an agriculturally compatible carrier.

Non-limiting examples of the above provided composition in practice are soluble powders, wettable granules, dry flowables, aqueous flowables, wettable dispersible granules, emulsifiable concentrates, aqueous suspensions, a fertilizer granule, a sprayable formulation, an agrochemical formulation. Thus, in another embodiment, an agricultural composition comprising the bacterial strain of current application is provided. “Agricultural composition” as used herein refers to a composition for agricultural purposes. Given that the composition is of use to promote plant growth and development, more particularly to promote the quantitative and/or qualitative yield of a leguminous plant, even more particularly to promote nitrogen fixation of a leguminous plant, most particularly to increase the protein content of soybeans, also a plant growth promoting composition is provided. Plant growth promoting refers to a promoting effect on the growth and development of the cultured plant or crop. Said cultured plant or crop is the plant or crop of interest and does not include unwanted plants. As described above, the composition or “plant growth promoting composition” herein provided can include a herbicide, if said herbicide is used to remove unwanted plants or prevent germination of seeds of unwanted plants. The composition, agricultural composition or plant growth promoting composition can also comprise a fertilizer, a micronutrient fertilizer material, an insecticide, a plant growth amendment, a fungicide, a molluscicide, an algicide, a bacterial inoculant, a fungal inoculant, or a combination thereof. In some cases, the fertilizer is a liquid fertilizer. Liquid fertilizer can include without limitation, ammonium sulfate, ammonium nitrate, ammonium sulfate nitrate, ammonium chloride, ammonium bisulfate, ammonium polysulfide, ammonium thiosulfate, aqueous ammonia, anhydrous ammonia, ammonium polyphosphate, aluminum sulfate, calcium nitrate, calcium ammonium nitrate, calcium sulfate, calcined magnesite, calcitic limestone, calcium oxide, hampene (chelated iron), dolomitic limestone, hydrate lime, calcium carbonate, diammonium phosphate, monoammonium phosphate, potassium nitrate, potassium bicarbonate, monopotassium phosphate, magnesium nitrate, magnesium sulfate, potassium sulfate, potassium chloride, sodium nitrates, magnesian limestone, magnesia, disodium dihydromolybdate, cobalt chlorid hexahydrate, nickel chloride hexahydrate, indole butyric acid, L-tryptophan, urea, urea-formaldehydes, urea ammonium nitrate, sulfur-coated urea, polymer-coated urea, isobutylidene diurea, K₂SO₄-2MgSO₄, kainite, sylvinite, kieserite, Epsom salts, elemental sulfur, marl, ground oyster shells, fish meal, oil cakes, fish manure, blood meal, rock phosphate, super phosphates, slag, bone meal, wood ash, manure, bat guano, peat moss, compost, green sand, cottonseed meal, feather meal, crab meal, fish emulsion or a combination thereof. The micronutrient fertilizer material can comprise boric acid, a borate, a boron frit, copper sulfate, a copper frit, a copper chelate, a sodium tetraborate decahydrate, an iron sulfate, an iron oxide, iron ammonium sulfate, an iron frit, an iron chelate, a manganese sulfate, a manganese oxide, a manganese chelate, a manganese chloride, a manganese frit, a sodium molybdate, molybdic acid, a zinc sulfate, a zinc oxide, a zinc carbonate, a zinc frit, zinc phosphate, a zinc chelate or a combination thereof. In a particular embodiment, said fertilizer or fertilizer material does not comprise insoluble selenium, selenium mineral, soluble selenium or salts thereof. The insecticide can include an organophosphate, a carbamate, a pyrethroid, an acaricide, an alkyl phthalate, boric acid, a borate, a fluoride, sulfur, a haloaromatic substituted urea, a hydrocarbon ester, a biologically-based insecticide, or a combination thereof. The herbicide can comprise a chlorophenoxy compound, a nitrophenolic compound, a nitrocresolic compound, a dipyridyl compound, an acetamide, an aliphatic acide, an anilide, a benzamide, a benzoic acid, a benzoic acid derivative, anisic acid, an anisic acid derivative, a benzonitrile, benzothiadiazinone dioxide, a thiocarbamate, a carmabate, carbanilate, chloropyridinyl, a cyclohexenone derivative, a dinitroaminobenzene derivative, a fluorodinitrotoluidine compound, isoxazolidinone, nicotinic acide, isopropylamine, an isopropulamine derivative, oxadiazolinone, a phosphate, a phthalate, a picolinic acid compound, a triazine, a triazole, a uracil, a urea derivative, endothall, sodium chlorate, or a combination thereof. The fungicide can comprise a substituted benzene, a thiocarbamate, an ethylene bis dithiocarbamate, a thiophthalidamide, a copper compound, an organomercury compound, an organotin compound, a cadmium compound, anilazine, benomyl, cyclohexamide, dodine, etridiazole, iprodione, metlaxyl, thiamimefon, triforine, or a combination thereof. The fungal inoculant can comprise a fungal inoculant of the family Glomeraceae, a fungal inoculant of the family Claroidoglomeraceae, a fungal inoculant of the family Acaulosporaceae, a fungal inoculant of the family Sacculospraceae, a fungal inoculant of the family Entrophosporaceae, a fungal inoculant of the family Pacidsproraceae, a fungal inoculant of the family Diversisporaceae, a fungal inoculant of the family Paraglomeraceae, a fungal inoculant of the family Archaeosporaceae, a fungal inoculant of the family Geosiphonaceae, a fungal inoculant of the family Ambisporacea, a fungal inoculant of the family Scutellosproaceae, a fungal inoculant of the family Dentiscultataceae, a fungal inoculant of the family Racocetraceae, a fungal inoculant of the phylum Basidiomycota, a fungal inoculant of the phylum Ascomycota, a fungal inoculant of the phylum Zygomycota, a fungal inoculant of the genus Glomus or a combination thereof. The bacterial inoculant can include a bacterial inoculant of the genus Rhizobium, another bacterial inoculant of the genus Bradyrhizobium, bacterial inoculant of the genus Mesorhizobium, bacterial inoculant of the genus Azorhizobium, bacterial inoculant of the genus Allorhizobium, bacterial inoculant of the genus Burkholderia, bacterial inoculant of the genus Sinorhizobium, bacterial inoculant of the genus Kluyvera, bacterial inoculant of the genus Azotobacter, bacterial inoculant of the genus Pseudomonas, bacterial inoculant of the genus Azosprillium, bacterial inoculant of the genus Bacillus, bacterial inoculant of the genus Streptomyces, bacterial inoculant of the genus Paenibacillus, bacterial inoculant of the genus Paracoccus, bacterial inoculant of the genus Enterobacter, bacterial inoculant of the genus Alcaligenes, bacterial inoculant of the genus Mycobacterium, bacterial inoculant of the genus Trichoderma, bacterial inoculant of the genus Gliocladium, bacterial inoculant of the genus Klebsiella, or a combination thereof.

Also, the application provides a combination comprising the bacterial strain of current application and at least one microorganism selected from the list consisting of Bacillus subtilis strain 713, Bacillus amyloliquefaciens MBI 600, Bacillus pumillus QST2808, Pseudomonas fluorescens, Trichoderma vireus, Pseudomonas putida, Trichoderma harzianum Rifai strain T22, Penicillium bilaii, Mesorhizobium, Azospirillum, Azotobacter vinelandii and Clostridium pasteurianum.

In another embodiment, an agricultural or plant growth promoting composition comprising the bacterial strain of current application and an agriculturally compatible carrier is provided.

### Coated Seeds

In a next aspect, a plant seed or plant propagule coated with a microbial population comprising the bacterial strain of current application is provided. This is equivalent as saying that a plant seed or plant propagule is provided, wherein said plant seed or propagule having applied to the surface of said seed or of said propagule, a culture, an enriched culture or a biological pure culture of the bacterial strain of current application. In one embodiment, said bacterial strain of current application is the Bradyrhizobium japonicum strain RHG_Soy_223 with deposit number LMG P-32018.

A “plant propagule” is any plant material for the purpose of plant propagation. Because of the totipotency of plants, any part of the plant may be used (e.g. a stem cutting, a leaf section, a portion of a root), though it is usually a highly meristematic part such as root and stem ends, buds, tubers, bulbs, rhizome, stolon or any plant part for vegetative reproduction. In sexual reproduction, a propagule is a seed or spore.

A “plant seed coated” or alternatively a “coated seed” as used in this application refers to a plant seed covered with a certain composition. This composition (i.e. the coating composition) can be a water composition or an oil composition or a polymer or any of the above described compositions comprising the bacterial strain of the application. “Coating” includes the most simple covering methods of dipping seeds or plant propagules in a microbial suspension or spraying seeds or propagules with a microbial suspension. In the latter case, the coating compositions are found to be film-forming, i.e. upon contacting with seeds or propagules they form a thin liquid film that adheres to the surface. “Coating” also includes rolling seeds/propagules in or dusting seeds/propagules with or brushing seeds/propagules with a powder comprising microorganisms, to more complex procedures as injecting plant seeds/propagules with a composition comprising microorganism or the use of complex coating layers including one or more adhesive, binder solvent and/or filler components. A person skilled in the art is familiar with a variety of conventional and more advanced methods to coat plants seeds (e.g. U.S. Pat. No. 5,113,619, EP0080999, WO1997036471, EP0010630, WO2006131213, WO2001045489, U.S. Pat. No. 4,465,017, EP2676536 which are here all incorporated as reference). The coating composition can include a number of ingredients, including but not limited to gelatin, a desiccant, water, tallow (e.g. to increase the release rate of any active ingredient in the composition), bulking agents (e.g. clay, vermiculite, perlite and/or bentonite to give more body to the liquid coating composition). Coating compositions which include bulking agents produce more rounded coated seeds. Such coated seeds are generally easier to plant when using mechanical planters. The concentration of the bulking agent can be up to about 50% of the solids by volume. As way of example of a liquid coating procedure, seeds or propagules are fed into one or more tanks containing the liquid coating composition. The seeds or propagules are transported from the tanks into a drying zone where forced air dries and solidifies the coating applied to the seeds. The seeds or propagules are dipped at least once and preferably at least twice in the liquid coating composition of the present invention. The dried coated seeds or propagules can be sowed or planted using standard sowing or planting machinery or by hand. In the alternative, the coated seeds or propagules can be stored for later application. If the temperature and humidity are relatively high or if prolonged storage is contemplated, it is desirable to place on the surface of the coating an inert material, preferably a powder material, such as, chalk or talcum powder. Such inert material reduces the tendency for the seed to stick together or agglomerate. The coating should cover more than 50%, more than 60%, more than 70%, more than 80%, more than 90, more than 95% of the surface area of the seeds or propagules. In some embodiments, after the coating procedure, the seeds should comprise at least one living cell of the isolated bacterial strain of current application. The coating layer can also consist of one or more components. These components can be additional plant growth promoting microorganisms but can also be fertilizers, biocontrol agents, or pesticides including fungicides, insecticides and herbicides. Non-limiting examples of these components are provided above. The coating composition can also include protective colloids, adhesives, thickening agents, thixotropic agents, penetrating agents, stabilizing agents, sequestering agents, fertilizers, anti-freeze agents, repellents, color additives, corrosion inhibitors, water-repelling agents, siccatives, UV-stabilizers, pigments, dyes or polymers.

In another embodiment, when used as a seed treatment, the bacterial strain of current application is applied at a rate of about 1×10² to about 1×10¹¹ cfu/seed or at a rate of about 1×10³ to about 1×10¹⁰ cfu/seed or at a rate of at least 1×10², at least 1×10³, at least 1×10⁴, at least 1×10⁵, at least 1×10⁶, at least 1×10⁷, at least 1×10⁸, at least 1×10⁹, at least 1×10¹⁰ or at least 1×10¹¹ cfu/seed. In yet another embodiment, for coating purposes seeds are treated with a bacterial solution of at least 1×10⁵ cfu of the bacterial strain of current application per ml, at least 1×10⁶ cfu of the bacterial strain of current application per ml, at least 1×10⁷ cfu of the bacterial strain of current application per ml, at least 1×10⁸ cfu of the bacterial strain of current application per ml, at least 1×10⁹ cfu of the bacterial strain of current application per ml, at least 1×10¹⁰ cfu of the bacterial strain of current application per ml or at least 1×10¹¹ cfu of the bacterial strain of current application per ml. After the coating procedure, the bacterial strain of current application is present on the seeds in a concentration of between 1×10⁴ and 1×10⁷ CFU, between 1×10⁵ and 5×10⁶ CFU per seed or at least 1×10⁵ CFU, at least 1×10⁶ CFU or at least 1×10⁷ CFU per seed.

In a particular embodiment, a plant seed refers to a seed of a leguminous plant. A leguminous plant or alternatively phrased a legume is referred in current application as a plant from the family Fabaceae (or Leguminosae). When used as a dry grain, the seed is also called a pulse. Leguminous plants are grown agriculturally, primarily for human consumption, for livestock forage and silage, and as soil-enhancing green manure. Well-known leguminous plants include beans (Phaseolus), soybeans (Glycine max), broad beans (Vicia faba), peas (Pisum sativum), chickpeas (Cicer arietinum), bitter vetch (Vicia ervilia), peanuts (Arachis hypogaea), lentils (Lens culinaris), lupins (Lupinus), mesquite (Prosopis), carob (Ceratonia siliqua), tamarind (Tamarindus indica), alfalfa (Medicago sativa), liquorice (Glycyrrhiza glabra) and clover (Trifolium sp.).

In another aspect, a method is provided of treating plant seeds, the method comprises the step of applying to said seeds an inoculum of the bacterial strain of the application. In one embodiment, said treating is coating. In another embodiment, said plant seeds are seeds from a leguminous plant, more particularly soybean.

### Applications

In a next aspect, the use of the Bradyrhizobium japonicum strain of the application or of a microbial population comprising it or of any of the previously described cultures is provided to increase or improve plant yield, more particularly agricultural yield.

“Yield” as used herein, generally refers to a measurable product from a plant, and more particularly to the amount or quality of harvestable plant material or plant-derived product. “Yield” is normally defined as the measurable produce of economic value of a crop. For crop plants, “yield” also means the amount and/or quality of harvested material per hectare or unit of production. Yield may be defined in terms of quantity or quality. The harvested material may vary from crop to crop, for example, it may be seeds, above ground biomass, roots, fruits, fibres, any other part of the plant, or any plant-derived product which is of economic value. The term “yield” also encompasses yield potential, which is the maximum obtainable yield. Yield may be dependent on a number of yield components, which may be monitored by certain parameters. These parameters are well known to persons skilled in the art and vary from crop to crop. The yield can be determined using any convenient method, for example, kilograms of plant product produced per hectare of planting or bushels or pound of plant product produced per acre of planting. The term “yield” also encompasses harvest index, which is the ratio between the harvested biomass over the total amount of biomass. The harvest index is relatively stable under many environmental conditions, and so a robust correlation between plant size and yield is possible. Yield and yield increase (in comparison to a control plant) can be measured in a number of ways, and it is understood that a skilled person will be able to apply the correct meaning in view of the particular embodiments, the particular crop concerned and the specific purpose or application concerned. The terms “enhanced yield” or “improved yield” or “increased yield” can be used interchangeable. As used herein, the term “enhanced yield” means any statistically significant improvement of one or more yield parameters selected from the group consisting of biomass yield, dry biomass yield, aerial dry biomass yield, underground dry biomass yield, fresh-weight biomass yield, aerial fresh-weight biomass yield, underground fresh-weight biomass yield, enhanced yield of harvestable parts, either dry or fresh-weight or both, either aerial or underground or both, enhanced yield of seeds, either dry or fresh-weight or both, either aerial or underground or both, improved nutrient use efficiency, improved seed set and harvest, improved protein content per seed, increased stress tolerance., increased efficiency of nodulation and/or nitrogen fixation, increased efficiency of carbon assimilation, improvement of seedling vigour/early vigour and/or enhanced efficiency of germination (under stressed or non-stressed conditions).

For example, yield refers to biomass yield, e.g. to dry weight biomass yield and/or fresh-weight biomass yield. Biomass yield refers to the aerial or underground parts of a plant, depending on the specific circumstances (test conditions, specific crop of interest, application of interest, and the like). In one embodiment, biomass yield refers to the aerial and underground parts. Biomass yield may be calculated as fresh-weight, dry weight or a moisture adjusted basis. Biomass yield may be calculated on a per plant basis or in relation to a specific area (e.g. biomass yield per acre/square meter/or the like). “Yield” can also refer to seed yield which can be measured by one or more of the following parameters: number of seeds or number of filled seeds (per plant or per area (acre, square meter or the like); seed filling rate (ratio between number of filled seeds and total number of seeds); number of flowers per plant; seed biomass or total seeds weight (per plant or per area (acre, square meter or the like); thousand kernel weight (TKW; extrapolated from the number of filled seeds counted and their total weight; an increase in TKW may be caused by an increased seed size, an increased seed weight, an increased embryo size, and/or an increased endosperm) and protein content of the harvested seeds. Other parameters allowing to measure seed yield are also known in the art. Seed yield may be determined on a dry weight or on a fresh weight basis, or typically on a moisture adjusted basis, e.g. at 15.5% moisture. For example, the term “increased yield” means that a plant, exhibits an increased growth rate, e.g. in the absence or presence of abiotic environmental stress, compared to the corresponding wild-type plant. An increased growth rate may be reflected inter alia by or confers an increased biomass production of the whole plant, or an increased biomass production of the aerial parts of a plant, or by an increased biomass production of the underground parts of a plant, or by an increased biomass production of parts of a plant, like stems, leaves, blossoms, fruits, and/or seeds. A prolonged growth comprises survival and/or continued growth of the plant, at the moment when the untreated control plant shows visual symptoms of deficiency and/or death.

In accordance with the invention, changes in different phenotypic traits may improve yield. For example, and without limitation, parameters such as floral organ development, seed number, seed weight, protein content of the seed, root initiation, root biomass, harvest index, leaf formation, phototropism, apical dominance, and fruit development, are suitable measurements of improved yield. Increased yield includes higher seed yields, higher protein content of the seed, higher fresh matter production, and/or higher dry matter production. Any increase in yield is an improved yield in accordance with the invention. For example, the improvement in yield can comprise a 0.1%, 0.2%, 0.5%, 0.8%, 1%, 3%, 5%, 10%, 15%, 20%, 30%, 40%, 50%, 60%, 70%, 80%, 90% or greater increase in any measured parameter compared to a mock situation. For example, an increase in the seed/acre yield of soy treated with the bacterial strain of the invention, as compared with the seed/acre yield from untreated soy cultivated under the same conditions, is an improved yield in accordance with the invention. Also an increased protein content/seed of soy treated with the bacterial strain of the invention, as compared with the protein content/seed from untreated soy cultivated under the same conditions, is an improved yield in accordance with the invention.

The yield of a plant can depend on the specific plant or crop of interest as well as its intended application (such as food production, feed production, processed food production, biofuel, biogas or alcohol production, or the like) of interest in each particular case. In one embodiment, yield can be calculated as harvest index (see definition above), harvestable parts weight per area (acre, square meter, or the like); and the like. Measurements of plant size in early development, under standardized conditions in a growth chamber or greenhouse, are standard practices to measure potential yield advantages conferred by the presence of plant growth promoting or nitrogen fixing bacteria.

When the plant treated with the bacterial strain of the application is a leguminous plant, increased yield means, in one embodiment, increased seed yield. Increased seed yield refers to a statistically significant increase or an at least 0.1%, 0.2%, 0.5%, 0.8%, 1%, 2%, 3%, 4%, 5%, 10%, 15%, 20%, 30%, 40%, 50%, 60%, 70%, 80%, 90% or more increase compared to an untreated but identical leguminous plant. In particular, increased yield for soy plants means increased seed yield, in particular for soy varieties used for feed or food. Non-limiting examples of soy varieties are Primus, Shouna, Bettina, Amarok, Lenka, Artemis and Hermes. Increased seed yield of soy refers in one embodiment to an increased seed size or weight, an increased number of seeds per pod, an increased number of pods per plant, improvement of seed composition or an increased protein content of the seeds. In a particular embodiment, increased protein content means obtaining a protein content of between 35 and 48%, of between 39 and 45%, between 40 and 44%, between 41 and 43% or of least 42% or of least 43% or of least 44%. The protein content of seeds, more particularly of soybeans can be measured by several methods known by the skilled person. The classical method for whole seed protein determination is the complete extraction of whole seed protein followed by nitrogen determination using either the Kjeldahl method (ISO 5683-2) or the Bradford assay based on a protein/nitrogen ratio of 6.25 (e.g. Yu et al 2016 Food Chem 196; James and Aijun et al 2016 Food Chem 194). Hence, the protein content can be calculated according to the formula: protein content=nitrogen content×6.25. Another non-limiting example of protein measurement is the combination of near infrared (NIR) spectroscopy and the Kjeldahl method. In a nutshell, dry seed material from a collection of samples is first analysed by NIR spectroscopy. Next, in order to calibrate the NIR spectroscopy data, protein content of a selection (e.g. 10%) of the samples covering the spectral variation of the complete set of samples is determined using the Kjeldahl method. Finally, based on the calibrated NIR spectroscopy data the protein content of all samples can be determined (Pannecoucque et al 2018 Eur J Agron 132; Pannecoucque et al 2018 J Agr Sci 156). In a particular embodiment of the invention, the protein content percentages herein disclosed are determined based on the Kjeldahl method (ISO 5683-2).

In another embodiment, increased seed yield of soy refers to an increased harvest of seeds per area, such as acre or hectare (ha). In a particular embodiment, increased seed yield means obtaining a seed yield of between 3.5 and 6 ton/ha, between 4 and 5.5 ton/ha or between 4.5 and 5 ton/ha or of at least 4 ton/ha, at least 4.5 ton/ha, at least 5 ton/h or of at least 5.5 ton/ha.

The above described increased or improved yield can be achieved in the absence or presence of stress conditions. The bacterial strain of the application is also provided to be of use to improve the adaptation of plants, more particular leguminous plants to cool growing conditions. More particularly to be of use to increase nitrogen fixation of the treated plant, more particularly leguminous plant in cool growing temperatures. How to measure nitrogen fixation in a plant is known by the person skilled in the art, e.g. as explained in Pannecoucque et al 2018 (Eur J Agron 132). In short, nitrogen (N) derived from the air is measured based on the different ratios of the stable nitrogen isotopes ¹⁵N: ¹⁴N in air and soil respectively.

In yet another embodiment, the use of the bacterial strain of the application is provided to increase cold tolerance of a plant. This solution is of great agricultural importance as low temperatures often significantly affect plant growth and crop productivity with crop losses as result (Xin and Browse 2001 Plant Cell Environ 23:893-902). Cold tolerance in plants is a very complex trait, involving many different metabolic pathways and cell compartments. Plants respond with changes in their pattern of gene expression and protein products when exposed to low temperatures. Plants differ in their tolerance to cold or chilling (0-17° C.) and freezing (<0° C.) temperatures. Plants of tropical and subtropical origins (e.g. soy) are highly sensitive to cold or chilling stress and are injured or killed by non-freezing low temperatures or have a reduced nitrogen fixing capacity. They exhibit various symptoms of chilling injury such as chlorosis, necrosis, or growth retardation. In contrast, plants from temperate climatic regions can be cold or chilling tolerant with variable degree and can be able to grow at such non-freezing cold temperatures. “Cold tolerance” or equivalently “chilling tolerance” or “low temperature tolerance” as used in current application is defined as the ability of a plant to tolerate low temperatures without or with limited injury, damage or yield drop, wherein said low temperatures are non-freezing temperatures. In one embodiment said low temperatures are temperatures between 5 and 20° C. or between 8 and 18° C. or between 10 and 15° C. In one embodiment, these temperatures are the temperatures of the soil or plant growth medium. Plants or plant roots are exposes to said low temperatures for at least 2 h, at least 4 h, at least 6 h or at least 8 h per day or said low temperatures are reached during at least a part of the day, for example during the night.

In particular embodiments, cold tolerance observed in plants that were treated with or were grown from seeds coated with the bacterial strain of current application leads to injury, damage or a drop in yield or nitrogen fixation due to low temperatures which is at least 10%, least 20%, least 30%, least 40%, least 50%, least 60%, least 70%, least 80%, least 90% or 100% less than the injury, damage or a drop in yield or nitrogen fixation observed in plants that were not treated with or were grown from seeds not coated with the bacterial strain of current application. In particular embodiments, the use of the bacterial strain of the application is provided to increase tolerance to non-freezing low temperatures in plants, more particularly leguminous plants, wherein said low temperatures are between 5 and 20° C. or between 8 and 18° C. or between 10 and 15° C. More particularly, increased tolerance to said non-freezing low temperatures in leguminous plants means increased tolerance of nitrogen fixation to said non-freezing low temperatures in leguminous plants, more particularly in soybean.

In a next aspect, a method is provided for enhancing growth, yield and/or cold tolerance of a plant comprising inoculating a plant growth medium with a microbial population, wherein said population comprises the bacterial strain of current application; and growing a plant in said plant growth medium; to enhance growth, yield and/or cold tolerance of said plant. In one embodiment, said yield is seed yield. In another embodiment, said enhancing yield is enhancing the protein content of seeds, more particularly of seeds of a leguminous plant. In one particular embodiment, said cold tolerance is cold tolerance of nitrogen fixation. Also provided is a method for enhancing nodulation or enhancing nitrogen fixation of a leguminous plant comprising inoculating a plant growth medium with a microbial population, wherein said population comprises the bacterial strain of current application and growing a leguminous plant in said plant growth medium to enhance nitrogen fixation of said plant. In one embodiment, said bacterial strains of current application is Bradyrhizobium japonicum RHG_Soy_223 with deposit number LMG P-32018. In another embodiment, said leguminous plant is soybean.

The term “enhancing nodulation” is defined herein as a statistically significant increase and/or an at least 5%, 10%, 20%, 30%, 40%, 60%, 70%, 80%, 90%, 100% or more increase in the number of nodules per root system, more particular per cm root system or per gram fresh or dry weight of the root system.

The term “inoculating” as used herein refers to introducing at least one bacterium into a plant growth medium. By way of example and without the intention to be limiting, said introduction can be performed using a liquid, a powder, a granule, a pellet. “Plant growth medium” is defined as any environment wherein plants can grow. Non-limiting examples of a plant growth medium are soil, sand, gravel, a polysaccharide, mulch, compost, peat moss, straw, logs, clay, or a combination thereof. A plant growth medium can also include a hydroculture system or an in vitro culture system. Hydroculture is the growing of plants in a soilless medium or an aquatic based environment, while an in vitro culture system refers to the growing of plants or explants on or in a recipient with synthetic medium, in sterile conditions, in a controlled environment and in reduced space. Explants refer to parts of a plant, from all the aerial part to isolated cells, as parts of leaves, of roots, seeds, bulbs, tubers, buds. The inoculation of said plant growth medium with a microbial population can be done before, during and/or after sowing or before, during and/or after the start of the plant growth cycle in case of hydroculture or in vitro culture. The inoculation can be performed once or multiple times during the plant growth cycle. In one embodiment, the microbial population is applied to the plant growth medium as a powder, as a pellet, as a granule or as a liquid.

The term “plant” as used herein encompasses whole plants and plant parts, including seeds, shoots, stems, leaves, roots (including tubers), bulbs, buds, flowers, and tissues and organs. The term “plant” also encompasses plant cells, suspension cultures, callus tissue, embryos, meristematic regions, gametophytes, sporophytes, pollen and microspores. Thus, in one embodiment, a method is provided for stimulating plant growth or yield comprising applying the microbial culture comprising the bacterial strain of current application to a plant, plant part, plant seed or to the plant growth medium. Unless otherwise specified, in the latter and further embodiments and aspects, “stimulating”, “enhancing”, “increasing” or “improving” refers to a statistically significant increase and/or an at least 5% increase or at least 6% increase or at least 7% increase or at least 8% increase or at least 9% increase or at least 10% increase or at least 12% increase or at least 15% increase or at least 20% increase or at least 25% increase or at least 30% increase or at least 50% increase or at least 75% increase or at least a 100% increase in the property being measured (e.g. plant growth, plant yield, nitrogen fixation) and compared to a mock or control situation.

Plants that are particularly useful in the methods of current application include in particular nitrogen fixing plants or leguminous plants. Non-limiting examples of leguminous plants are acacia (genus Acacia), alfalfa (Medicago sativa), almendro (Dipteryx oleifera), bean (genus Phaseolus), common bean (P. vulgaris), green bean (P. vulgaris), lima bean (P. lunatus), scarlet runner bean (P. coccineus), bird's-foot trefoil (Lotus corniculatus), bush clover (genus Lespedeza), broom (genus Cytisus), carob (Ceratonia siliqua), chickpea (Cicer arietinum), clover (genus Trifolium), cowpea (Vigna unguiculata), crown vetch (Securigera varia), fenugreek (Trigonella foenum-graecum), honey locust (Gleditsia species), hyacinth bean (Lablab purpureus), indigo (genus Indigofera), jícama (Pachyrhizus erosus), kakabeak (genus Clianthus), Kentucky coffee tree (Gymnocladus dioica), kidney vetch (Anthyllis vulneraria), kudzu vine (Pueraria montana), laburnum (genus Laburnum), golden chain (L. anagyroides), genus Lathyrus, beach pea (L. japonicus), sweet pea (L. odoratus), lentil (Lens culinaris), licorice (Glycyrrhiza glabra), locoweed (Astragalus and Oxytropis species), locust (genus Robinia), logwood (Haematoxylum campechianum), lupine (genus Lupinus), Texas bluebonnet (L. texensis and L. subcarnosus), mesquite (genus Prosopis), mimosa (genus Mimosa), sensitive plant (M. pudica), narra (Pterocarpus species), pagoda tree (Styphnolobium japonicum), palo verde (genus Parkinsonia), pea (Pisum sativum), peanut (Arachis hypogaea), redbud (genus Cercis), rosary pea (Abrus precatorius), royal poinciana (Delonix regia), senna (genus Senna), silk tree (genus Albizia), smoke tree (Dalea spinosa), soybean (Glycine max), suicide tree (Tachigali versicolor), sunn hemp (Crotalaria juncea), tamarind (Tamarindus indica), vetch (genus Vicia), broad bean (V. faba), wisteria (genus Wisteria). In a particular embodiment, said leguminous plant is selected from the list consisting of alfalfa (Medicago sativa), bean (genus Phaseolus), common bean (P. vulgaris), green bean (P. vulgaris), lima bean (P. lunatus), scarlet runner bean (P. coccineus), chickpea

(Cicer arietinum), clover (genus Trifolium), cowpea (Vigna unguiculata), fenugreek (Trigonella foenum-graecum), genus Lathyrus, beach pea (L. japonicus), sweet pea (L. odoratus), lentil (Lens culinaris), licorice (Glycyrrhiza glabra), pea (Pisum sativum), peanut (Arachis hypogaea), soybean (Glycine max), tamarind (Tamarindus indica), vetch (genus Vicia) and broad bean (V. faba). In a most particular embodiment, said leguminous plant is soybean (Glycine max), bean (Phaseolus sp.), lentils (Lens culinaris), chickpea (Cicer arietinum), clover (genus ), cowpea (Vigna unguiculata) or Lathyrus.

In another aspect, a method for enhancing growth, yield and/or cold tolerance of a plant is provided, wherein said method comprises growing coated seeds of a plant, wherein said seeds are coated with a microbial population comprising the isolated bacterial strain of current application, to obtain enhanced growth, yield and/or cold tolerance of said plant. In one embodiment, said yield is seed yield or amount of seeds (e.g. expressed in tons) per acre or hectares. In another embodiment, said enhancing yield is enhancing the protein content of seeds, more particularly of seeds of a leguminous plant. Also provided is a method for enhancing nodulation or enhancing nitrogen fixation of a leguminous plant, wherein said method comprises growing coated seeds of said plant, wherein said seeds are coated with a microbial population comprising the isolated bacterial strain of current application. In some embodiments, after the coating procedure, the seeds should comprise at least 1×10⁴, 1×10⁵ or 1×10⁶ living cells or spores of the bacterial strain of current application.

An “effective amount” refers to an amount sufficient to effect beneficial or desired results. In a non-limiting example, an “effective amount” leads to a statistically significant increase of plant growth and/or biomass and/or yield and/or cold tolerance and/or protein content of seed and/or nitrogen fixation as compared to the growth, biomass and/or yield and/or cold tolerance and/or protein content of seed and/or nitrogen fixation of the control plant. An effective amount can be administered in one or more administrations. A “control plant” as used in current application provides a reference point for measuring changes in phenotype of the subject plant and may be any suitable plant cell, seed, plant component, plant tissue, plant organ or whole plant. A control plant may comprise for example a plant or cell which is genetically identical to the subject plant or cell but which is not exposed to the same treatment (e.g. administration of the bacterial strain of current application) as the subject plant or cell.

In another embodiment, a method is provided for enhancing nutrient uptake and/or nutrient use efficiency of a plant, said method comprising growing coated seeds of a plant, wherein said seeds are coated with a microbial population comprising the bacterial strain of current application, to obtain enhanced nutrient uptake and/or nutrient use efficiency of said plant. In another embodiment, a method is provided for enhancing nodulation or enhancing the nitrogen fixating capacity of a plant, said method comprising growing coated seeds of a plant, wherein said seeds are coated with a microbial population comprising the bacterial strain of current application, to obtain enhanced nodulation or enhanced a nitrogen fixating capacity of said plant.

In yet another aspect, a method for enhancing growth, yield and/or cold tolerance of a plant is provided comprising:
growing a plant in an environment that supports plant growth; and
administering a sprayable formulation to said environment or to said plant, said formulation comprising the bacterial strain of current application;
to obtain enhanced growth, yield and/or cold tolerance of said plant. In one embodiment, said yield is seed yield. In another embodiment, said enhancing yield is enhancing the protein content of seeds of said plant, more particularly a leguminous plant. Also a method for enhancing nodulation or enhancing nitrogen fixation of a leguminous plant is provided, comprising the steps of growing said plant in an environment that supports plant growth and administering a sprayable formulation to said environment or to said plant, said formulation comprising the bacterial strain of current application. In a particular embodiment, said method is provided wherein said strain is Bradyrhizobium japonicum RHG_Soy_223 with deposit number LMG P-32018.

A “sprayable formulation” as used herein is an agrochemical or a biological solution that can be sprinkled on a plant or soil. The formulation is composed in such a way that the active ingredients can be absorbed by the above-ground tissue of a plant or is available for the plant roots when administered to the soil. The above disclosed methods thus also includes irrigation with a liquid comprising the bacterial strain of current application. “Irrigating” or “irrigation” as used herein refers to the method in which water or other liquids are supplied to plants at regular intervals. Irrigation includes but is not limited to “localized irrigation” (i.e. a system where water is distributed under pressure through a piped network, in a pre-determined pattern, and applied as a small discharge to each plant or adjacent to it. “Drip (or micro) irrigation”, also known as “trickle irrigation” (i.e. a system where water falls drop by drop just at the position of roots or near the root zone of plants) and “sprinkler irrigation” (i.e. a system where water is distributed by overhead sprinklers) belong to this category of irrigation methods. In “sprinkler irrigation”, sprinklers can also be mounted on moving platforms connected to the water source by a hose. Automatically moving wheeled systems known as traveling sprinklers may irrigate areas such as small farms, sports fields, parks and pastures unattended. Most of these utilize a length of polyethylene tubing wound on a steel drum. As the tubing is wound on the drum powered by the irrigation water or a small gas engine, the sprinkler is pulled across the field. When the sprinkler arrives back at the reel the system shuts off. This type of system is known to most people as a “waterreel” traveling irrigation sprinkler.

Hence, in various embodiments, a method is provided for enhancing growth, yield and/or cold tolerance of a plant, said method comprising:
growing said plant in an environment that supports plant growth;
irrigating said environment using a liquid solution comprising the bacterial strain of current application;
to obtain enhanced growth, yield and/or cold tolerance of said plant.

In one embodiment, said yield is seed yield. In another embodiment, enhancing yield is enhancing the protein content of seeds of said plant, more particularly the seeds of a leguminous plant. Also provided is a method to increase nodulation or the nitrogen fixation of a leguminous plant comprising the steps of growing said plant in an environment that supports plant growth and irrigating said environment using a liquid solution comprising the bacterial strain of current application.

In particular embodiments, when used as a soil treatment, the bacterial strain of current application can be applied as a soil surface drench, injected and/or applied in-furrow or by mixture with irrigation water. The rate of application for drench soil treatments, which may be applied at planting, during or after seeding, or after transplanting and at any stage of plant growth, is about 1×10¹¹ to about 8×10¹² cfu per acre. In some embodiments, the rate of application is about 1×10¹² to about 8×10¹² cfu per acre. The rate of application for in-furrow treatments, applied at planting, is about 2.5×10¹⁰ to about 5×10¹¹ cfu per 1000 row feet. In some embodiments, the rate of application is about 6×10¹⁰ to about 4×10¹¹ cfu per 1000 row feet. Those of skill in the art will understand how to adjust rates for broadcast treatments (where applications are at a lower rate but made more often) and other less common soil treatments.

In some embodiments, when the bacterial strain of current application is applied as microbial population or bacterial population or solution or culture or agricultural composition or sprayable formulation, the number of colony forming units (cfu) per milliliter (ml) of said bacterial strain of current application in the microbial populations or bacterial populations or solutions or cultures or agricultural compositions or sprayable formulations will be at least 1×10⁶ cfu/ml or at least 1×10⁷ cfu/ml or at least 1×108 cfu/ml or at least 1×10⁹ cfu/ml or at least 2×10⁹ cfu/ml or at least 3×10⁹ cfu/ml or at least 4×10⁹ cfu/ml or at least 5×10⁹ cfu/ml or at least 6×10⁹ cfu/ml or at least 7×10⁹ cfu/ml or at least 8×10⁹ cfu/ml or at least 9×10⁹ cfu/ml or at least 1×10¹⁰ cfu/ml or at least 2×10¹⁰ cfu/ml or at least 3×10¹⁰ cfu/ml or at least 4×10¹⁰ cfu/ml or at least 5×10¹⁰ cfu/ml or at least 6×10¹⁰ cfu/ml or at least 7×10¹⁰ cfu/ml or at least 8×10¹⁰ cfu/ml or at least 9×10¹⁰ cfu/ml or at least 1×10¹¹ cfu/ml or at least 2×10¹¹ cfu/ml or at least 3×10¹¹ cfu/ml or at least 4×10¹¹ cfu/ml or at least 5×10¹¹ cfu/ml or at least 6×10¹¹ cfu/ml or at least 7×10¹¹ cfu/ml or at least 8×10¹¹ cfu/ml or at least 9×10¹¹ cfu/ml or at least 1×10¹² cfu/ml or at least 1×10¹³ cfu/ml or at least 1×10¹⁴ cfu/ml.

Additional to the above detailed description of the invention, terminology as used in describing the aspects of the invention is described in the following sections. In all herein described aspects and embodiments-unless specified differently-“enhance” or “increase” or “improvement” refers to a statistically significant increase and/or an at least 1%, 2%, 3%, 4% or 5% increase or at least 6% increase or at least 7% increase or at least 8% increase or at least 9% increase or at least 10% increase or at least 15% increase or at least 20% increase or at least 25% increase or at least 30% increase or at least 50% increase or at least 75% increase or at least a 100% increase in the property being measured and compared to a control situation. Said control situation is a mock situation wherein the plant, plant seed or other plant part was not treated with the microbial population or bacterial strain herein disclosed. The skilled person is aware how a scientifically sound mock situation should be set up. “Treated” as used herein can be direct treatment (e.g. coating seeds or spraying plants) and/or indirect treatment (e.g. providing the substrate wherein the plant is growing with a bacterial population).

The term “statistically significant” or “statistically significantly” different is well known by the person skilled in the art. Statistical significance plays a pivotal role in statistical hypothesis testing. It is used to determine whether the null hypothesis should be rejected or retained. It states that the results are obtained because of chance and are not supporting a real change or difference between two data sets. The null hypothesis is the default assumption that what one is trying to prove did not happen. In contrast the alternative hypotheses states that the obtained results support the theory being investigated. For the null hypothesis to be rejected (and thus the alternative hypothesis to be accepted), an observed result has to be statistically significant, i.e. the observed p-value is less than the pre-specified significance level α. The ρ stands for probability and measures how likely it is that the null hypothesis is incorrectly rejected and thus that any observed difference between data sets is purely due to chance. In most cases the significance level α is set at 0.05.

“Microbial” as used herein refers to microorganisms, wherein said microorganisms can include bacteria, archaebacteria, fungi, yeasts, mycorrhiza, microscopic eukaryotes (e.g. protozoa and algae), viruses, viroids or a combination thereof. A “microbial population” as used herein can thus refer to a synthetic or artificial collection of different microorganisms with distinct geographical origins. In various more particular embodiments of this application, “microbial” refers to “bacterial”. For the purpose of current application, the term “bacterium” or “bacteria” includes any prokaryotic organism that does not have a distinct nucleus. While being both part of the group of microorganisms, bacteria and fungi are clearly distinct. The term “fungi” or “fungus” includes a wide variety of nucleated spore-bearing organisms that are devoid of chlorophyll.

In order to reconstruct the evolutionary relationships and sequence identity of one bacterial isolate to another, phylogenetic approaches are used standardly exploiting the 16S rRNA sequence or a portion of the 16S rRNA sequence of the bacteria, although any other sequence or the entire genome of the microorganisms to be analyzed can also be used. In microbiology, “16S rRNA sequence” refers to the sequence derived by characterizing the nucleotides that comprise the 16S ribosomal RNA gene(s). The bacterial 16S rRNA is approximately 1500 nucleotides in length. In this application “sequence similarity”, “sequence identity” and “sequence homology” are interchangeably used. The term “sequence identity” as used herein refers to the extent that sequences are identical on a nucleotide-by-nucleotide basis over a window of comparison. Thus, a “percentage of sequence identity” is calculated by comparing two optimally aligned sequences over the window of comparison, determining the number of positions at which the identical nucleic acid base (e.g., A, T, C, G, I) occurs in both sequences to yield the number of matched positions, dividing the number of matched positions by the total number of positions in the window of comparison (i.e., the window size), and multiplying the result by 100 to yield the percentage of sequence identity. A gap, i.e., a position in an alignment where a residue is present in one sequence but not in the other is regarded as a position with non-identical residues. Determining the percentage of sequence identity can be done manually, or by making use of computer programs that are available in the art. Examples of useful algorithms are PILEUP (Higgins & Sharp, CABIOS 5:151 (1989), BLAST and BLAST 2.0 (Altschul et al. J. Mol. Biol. 215:403 (1990). Software for performing BLAST analyses is publicly available through the National Center for Biotechnology Information (http://www.ncbi.nlm.nih.gov/).

The term “plant growth promoting” as used herein, refers to a promoting effect on a wide range of growth and development properties of cultured plants or crops, including but not limited to increased root development, increased leaf area, increased plant yield, increased fresh or dry weight, increased seed yield, increased seed germination, increased photosynthesis, increase in accumulated biomass of the plant, increased nitrogen fixation or increased efficiency of nutrients such as nitrogen, phosphorus or potassium.

“CFU” or “cfu” as used herein refers to colony-forming unit. This unit is well-known by the person skilled in the art of microbiology (as well as the methodology how to determine the number of colony-forming units) and is used to estimate the number of viable bacteria or fungal cells in a sample. “Viable” is defined as the ability to multiply via binary fission under controlled conditions. Counting with colony-forming units requires culturing the microbes and counts only viable cells, in contrast with microscopic examination which counts all cells, living or dead.

The present invention is described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated. Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

The terms or definitions provided herein are solely to aid in the understanding of the invention. Unless specifically defined herein, all terms used herein have the same meaning as they would to one skilled in the art of the present invention. Practitioners are particularly directed to Michael R. Green and Joseph Sambrook, Molecular Cloning: A Laboratory Manual, 4th ed., Cold Spring Harbor Laboratory Press, Plainsview, New York (2012); and Ausubel et al., Current Protocols in Molecular Biology (Supplement 47), John Wiley & Sons, New York (1999), for definitions and terms of the art. The definitions provided herein should not be construed to have a scope less than understood by a person of ordinary skill in the art.

It is to be understood that although particular embodiments, specific configurations as well as materials and/or molecules, have been discussed herein for cells and methods according to the present invention, various changes or modifications in form and detail may be made without departing from the scope and spirit of this invention. The Examples described below are provided to better illustrate particular embodiments, and they should not be considered limiting the application. The application is limited only by the claims.

### EXAMPLES

### Example 1. Trapping Native Soybean Nodulators

To ensure protein rich beans, soy plants have to establish a symbiosis with nitrogen-fixing bacteria in their root nodules. Currently, the use of commercial inoculants in Belgian and North-West European soybean agriculture in general is insufficient for a reliable qualitative yield. To solve this problem, we initiated a nodulation trap experiment during which three early-maturing soybean cultivars were grown in natural conditions and under greenhouse conditions in 107 garden soils to seek nodulating endogenous rhizobia. These three early-maturing cultivars (i.e. Primus, Bettina and Shouna) exhibit differences in oil and protein content, thousand kernel weight, disease tolerance, among others (Pannecoucque et al 2018). In about 20% of the soils, plants with root nodules or nodule-like structures were obtained. A large diversity in nodule size, shape and position on the root system of the plants was observed. The majority of the nodules were small, irregularly shaped or located at root branching points. Their white/brown colour indicated that they might be non-fixing. However, in other soils regularly shaped nodules were obtained with a light pink or red colour, which is the colour of leghaemoglobin, demonstrating active nitrogen fixation in the nodules.

### Example 2. Identification and Isolation of Endogenous Rhizobial Bacteria

In order to isolate and identify individual rhizobial bacterial strains residing in the soybean nodules, we crushed and plated out the juice of one nodule (per plant per cultivar per experiment) on YMA media allowing specific growth of rhizobia as well as other bacteria. For each nodule, one to eight bacterial isolates were obtained and their bacterial taxonomy was determined using 16S rRNA PCR sequencing. From more than 200 bacterial isolates, nine strains belonging to the genera Bradyrhizobium , Neorhizobium and Rhizobium were selected.

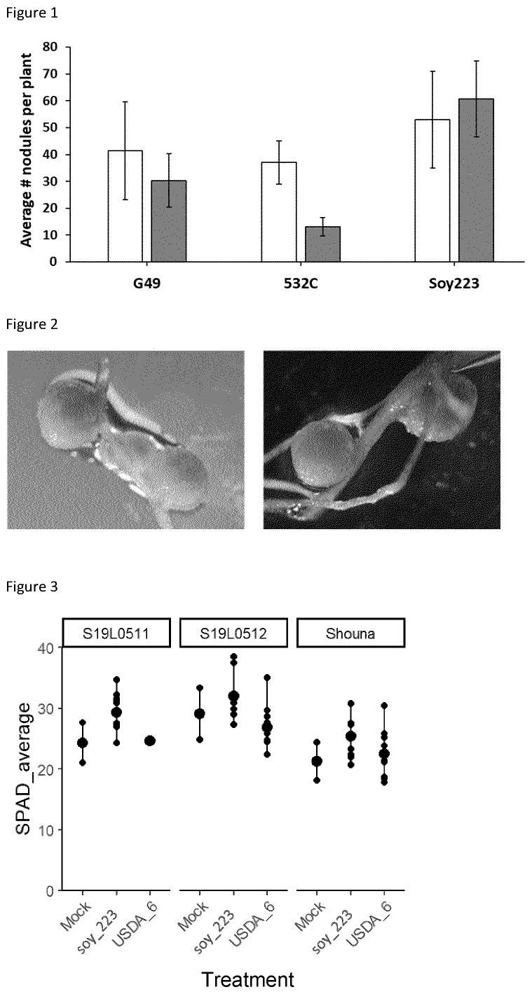

To confirm the nodulation potential of the isolated and selected strains, several repeated inoculation experiments on three soybean cultivars grown in sterile vermiculite under growth room conditions (16 h light/8 h dark at 22° C.) were performed. Plants were watered twice a week with nitrogen-poor SOLi solution (Blondon 1964 Rev Gén Bot 71). After one week, plants were inoculated with 1 ml of the respective bacteria at OD=0.01. After four weeks, nodule presence and colour were assessed. Inoculation with two commercial inoculants as positive controls (i.e. B. japonicum G49 sold under brand name Biodoz and B. japonicum 532C commercially available under brand name HiStick) resulted in large red nitrogen-fixing nodules on all soybean cultivar roots (FIG. 1). While all isolates induced nodules on minimum one of the cultivar roots, only one Bradyrhizobium strain repeatedly resulted in nodules in all experiments (FIG. 1). Moreover, the nodules were always red and in a large quantity present on the roots of all three cultivars (FIG. 2). A further phylogenetic analysis revealed that the strain belonged to the Bradyrhizobium japonicum species. The newly isolated B. japonicum is from here on referred to as B. japonicum_RHG_Soy_223.

### Example 3. B. japonicum RHG_Soy_223 is a Novel Strain

BLAST analyses based on the 16S rRNA sequence of B. japonicum RHG_Soy_223revealed 100% similarity with several known Bradyrhizobium japonicum strains, e.g. Bradyrhizobium strain USDA6 (Kaneko et al 2011 Genes 2), strain E10⁹ (Torres et al 2015 Genome Announcements 3) and the SEMIA strains 5086, 5081, 5057, 5052, 5051, 5039, 593, 579, 5079, 5038, 5029.

Next, the whole genome was sequenced, reads were assembled and contigs annotated. Different loci of the B. japonicum RHG_Soy_223 strain were BLASTed (i.e. atpD (ATP synthase subunit beta), recA (Protein recA), GyraseB, asd (aspartate-semialdehyde dehydrogenase), gnd (6-phosphogluconate dehydrogenase), sucA (2-oxoglutarate dehydrogenase E1) and zwf (glucose-6-phosphate 1-dehydrogenase) (Berkum et al 2006) Bacteriol 188:5570-5577, doi: 10.1128/JB.00335-06)). For all tested genes, 100% identity was found between B. japonicum RHG_Soy_223 and three known B. japonicum strains: E109, SEMIA 5038 and USDA6.

Finally, the full genome of B. japonicum RHG_Soy_223 was compared to those of several publicly available B. japonicum strains and the average nucleotide identity (ANI) values were determined (Table 1). The ANI value is a measure of nucleotide-level genomic similarity between the coding regions of two genomes. Although B. japonicum RHG_Soy_223 is also on full genome level highly similar to publicly available B. japonicum strains, B. japonicum RHG_Soy_223 can be distinguished from the known strains and is thus a novel strain.

TABLE 1
Whole Genome Sequence comparison with
B. japonicum RHG_Soy_223

| Strain | % identity |
|---|---|
| E109 | 99.97% |
| SEMIA 5038 | 99.97% |
| USDA6 | 99.97% |
| NBRC14783T | 99.98% |
| 5873 | 99.96% |
| FN1 | 99.96% |

### Example 4. Comparison Between B. japonicum RHG_Soy_223 and USDA6

Next, we selected one of the closely related and publicly available B. japonicum strains to set up comparative experiments with B. japonicum RHG_Soy_223. Other strains indicated in Table 1 were not commercially or publicly available for testing when the below comparative experiments were performed.

### 4.1 Soybean Inoculation

In a first experiment, 3 soybean cultivars (i.e. S19L0511, S19L0512 and Shouna) were grown in the greenhouse and inoculated with a mock control, with B. japonicum RHG_Soy_223 or with the USDA6 strain as specified in the Experimental procedures. At 4 weeks the Soil Plant Analysis Development (SPAD) value was determined (FIG. 3). SPAD is a non-destructive measurement of chlorophyll content from the last expanded leaf of a plant and one of the most commonly used diagnostic tools to measure crop nitrogen status. The measurement is based on the ratio of radiation transmittance from two wavelengths through the leaf, e.g. the transmittance of red (650 nm) and infrared (940 nm) radiation (Uddling et al 2007 Photosynth Res 91).

On all soybean cultivars tested, the plants inoculated with the B. Japonicum RHG_Soy_223 strain showed the highest SPAD values, indicating an improved nitrogen status.

### 4.2 Bioscreen Experiments

The B. japonicum strains RHG_Soy_223 and USDA6 were grown on YMA plates (0.2 g K₂HPO₄, 0.2 g MgSO₄, 10 g mannitol, 0.3 g yeast extract, 0.05 g NaCl, 15 g bacteriological agar in distilled water adjusted to pH 7 for 1 litre of YMA) at 30° C. for 3 days, then tubes with YMB medium were inoculated and grown for 3 days at 30° C. The final OD's were measured and adjusted to 1.0. For each strain 5 repeats of a 100-fold dilution in YMB medium were transferred to a Honeycomb plate designed for the Bioscreen C device (Growth Curves USA). The device performs an OD measurement every 15 minutes, 1 temperature can be set per run.

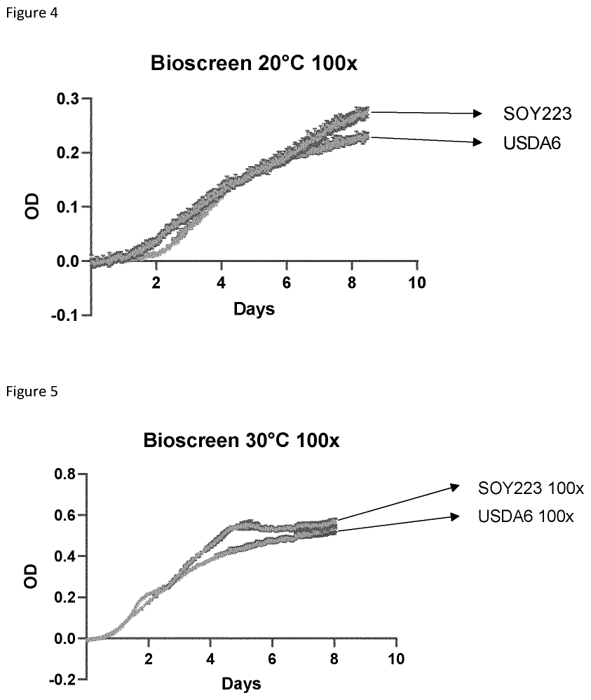

In a first experiment the growth of the Bradyrhizobia was checked at 20° C. It was surprisingly found that strain RHG_Soy_223 has an accelerated growth at 20° C. compared to USDA6 (FIG. 4). This is of special importance to allow a symbiotic interaction with soybean early in the growing season in North-West European regions. Interestingly an improved growth of RHG_Soy_223 compared to USDA6 could also be observed at 30° C. (FIG. 5).

### Experimental Procedures for the Above Examples

### Nodulation

Soybean seeds were surface sterilized first with 70% ethanol and then with a bleach solution (29 ml sterile water, 15 ml NaClO, 12-13% (v/v) stock solution, and 1 ml Tween 20), where after they were washed five times for 15 min with sterile water and allowed to germinate for 4 days in the dark at room temperature. Seedlings of each cultivar were sown in sterilized vermiculite in 13-cm round pots and grown under a 16-h light/8-h dark photoperiod at 22° C. in the greenhouse. Plants were watered twice a week with nitrogen-poor SOLi solution. After one week, plants were inoculated with 1 ml of the respective bacteria at OD=0.01. Four weeks after inoculation, the nodule presence was assessed and the nodules were photographed. These screening experiments were repeated independently seven times for each candidate isolate. The commercial Bradyrhizobium inoculants, G49 and 532C, were used as positive controls for nodules. Non-inoculated mock plants were used as negative controls.

### Example 5. Comparison of Various B. japonicum Strains' Survival in a Commercial Coating

Some seeds, such as seeds of small-seeded forage legumes, including soy seeds, benefit from mixing inoculant strains with adhesives, also known as stickers or glue, in order to have sufficient inoculant per unit of seed-surface area.

G49 is a commercial B. japonicum strain known to increase nodulation in soy. The survival of RHG_Soy_223 was compared to the survival of said commercial G49 strain, specifically in a commercial seed coating comprising a commercial seed sticker.

To this end, the B. japonicum strain RHG_Soy_223 and commercial strain B. japonicum G49 were grown from a single colony in tubes with YMB media for 3 days, subsequently transferred to and grown in Erlenmeyers with YMB for another 3 days. The cultures were centrifuged and pellets mixed as indicated by the manufacturer with the commercial sticker (4:1 bacteria: sticker). The mixture was added to soy seeds on a seed coater and subsequently, the seeds were stored at 25° C. Every other day, including day 0—the day of seed coating-, 10 seeds were taken from each treatment and re-suspended in a MgSO₄ solution. The bacterial suspension was diluted and plated on YMA plates. Colonies were counted and the original CFU concentration was determined, based on the performed dilutions.

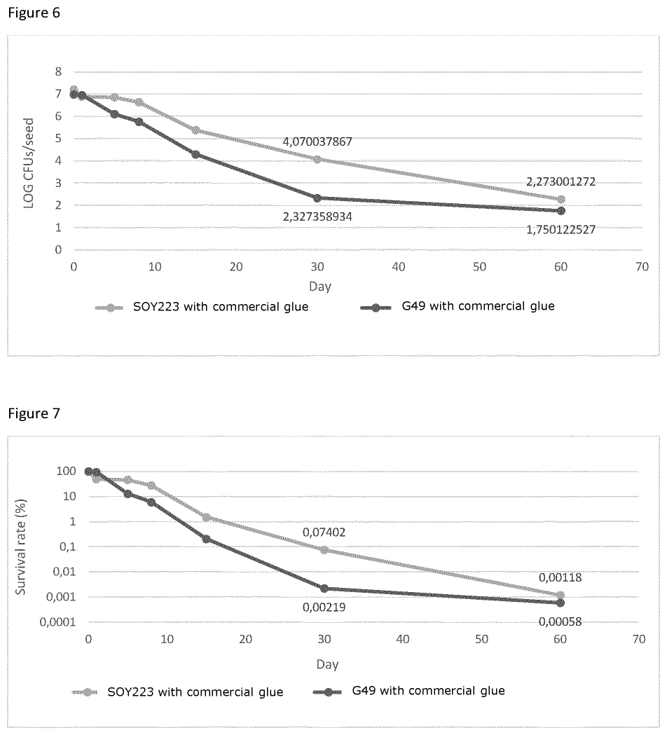

After 30 days, for RHG_Soy_223 on average about 10\hat{ }4.07 CFUs/seed were counted, while for G49 this was on average about 10\hat{ }2.33 CFUs/seed, thus the average count per seed was over 200 times lower for G49 than for RHG_Soy_223 (FIG. 6). In terms of survival rate (%), this was 0.074% for RHG_Soy_223 compared to only 0.002% for G49 (FIG. 7).

After 60 days, the difference in the average count of CFUs/seed was still over 3 times higher for RHG_Soy_223 than for G49 (FIG. 6). In terms of survival rate (%) this was 0.0012% for RHG_Soy_223 compared to only 0.0006% for G49 (FIG. 7).

### Example 6. Growth of Various B. japonicum Strains in Control and Low-Temperature Conditions

The B. japonicum strains RHG_Soy_223, and closely related B. japonicum strains G49, USDA6, and 532C, were each grown from a single colony in tubes with 5 ml YMB media, subsequently incubated in a shaking incubator at 20° C. or 30° C. for 3 days. 3 dpi, 1 ml of the culture was added to an Erlenmeyer with 25 ml of YMB in duplicate. The optical density at 600 nm was determined for each Erlenmeyer at day 0 and the cultures were incubated at either 20 or 30° C., following the OD for 7 days. The measured ODs were normalized to the values of day 0.

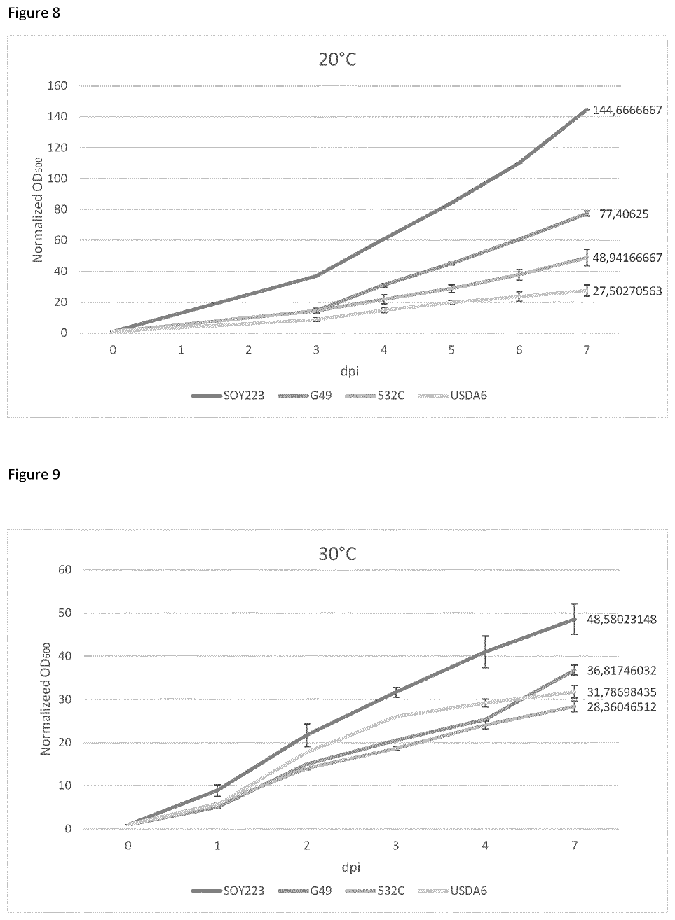

RHG_Soy_223's growth clearly outperforms the growth of the three other closely related strains, each day post-inoculation, both at 20° C. and 30° C. Results are shown in FIGS. 8 (20° C.) and 9 (30° C.).

### Example 7. Chlorophyll Content and Number of Nodules in Soy Grown in Low-Temperature Conditions, Inoculated with Different B. japonicum Strains

Three non-inoculated seeds of the soy variety Artemis were planted per pot containing a mix of potting soil, sand and perlite. After sowing, each seed was inoculated with 0.333 ml of a 0.01 OD₆₀₀ suspension of bacterial strains USDA6, G49, or RHG_Soy_223 grown in YMB as described above. The negative control was inoculated with 0.333 ml of YMB. 5 pots per treatment were randomly placed in growth chambers with 12 h/12 h light/darkness and 22/10° C. regimes. 7 days post-sowing, 2 plantlets were removed from each pot, leaving 1 in the soil. 33 days post-sowing, SPAD values were measured in the youngest fully-developed leaf of each plant, and the nodule number was determined.

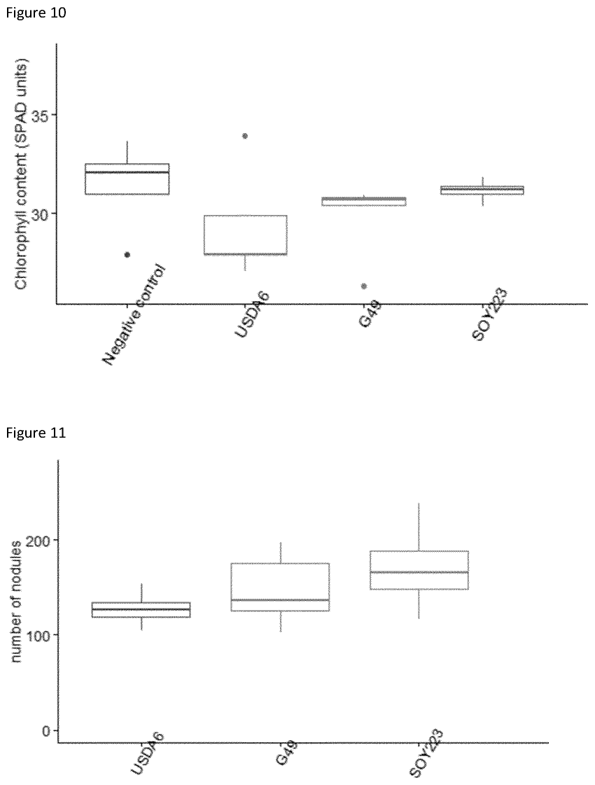

Results are shown in FIGS. 10 (chlorophyll content—SPAD values) and 11 (number of nodules). The inoculated plants have a lower chlorophyll content than the non-inoculated plants. This shows that at these low temperatures, the interaction with the bacteria is energetically expensive for the plant. Interaction with RHG_Soy_223, however, seems to be the least energy-consuming for the plants (FIG. 10). FIG. 11 shows that RHG_Soy_223 is able to nodulate better than the other 2 strains in these low-temperature conditions.

1-22. (canceled)

23. A method for enhancing yield and/or nodulation of a leguminous plant or enhancing the protein content of the seeds of a leguminous plant, the method comprising the steps of:
inoculating a plant growth medium with a microbial population, said microbial population comprising a Bradyrhizobium japonicum strain; and
growing the leguminous plant in said plant growth medium, wherein the Bradyrhizobium japonicum strain has a deposit accession number LMG P-32018 or has at least 99.90% genomic sequence identity with a strain having the deposit accession number LMG P-32018, and wherein the Bradyrhizobium japonicum strain has retained plant growth and/or yield characteristics at temperatures between 10 and 25° C. of the deposited strain LMG P-32018.

24. The method of claim 23, wherein the microbial population is applied to the plant growth medium as a powder, as a pellet, as a granule or as a liquid.

25. A method for enhancing yield and/or nodulation of a leguminous plant or enhancing the protein content of the seeds of a leguminous plant, said method comprising growing a coated plant seed to obtain enhanced yield and/or nodulation of said leguminous plant or an enhanced protein content of the seeds of said leguminous plant, wherein the coated plant seed is coated with a Bradyrhizobium japonicum strain that has a deposit accession number LMG P-32018 or that has at least 99.90% genomic sequence identity with a strain having the deposit accession number LMG P-32018, and wherein the Bradyrhizobium japonicum strain has retained plant growth and/or yield characteristics at temperatures between 10 and 25° C. of the deposited strain LMG P-32018.

26. A method for enhancing yield and/or nodulation of a leguminous plant or enhancing the protein content of the seeds of a leguminous plant comprising:
growing said leguminous plant in an environment that supports plant growth; and
administering a sprayable formulation to said environment or to said leguminous plant, said formulation comprising a Bradyrhizobium japonicum strain
to obtain enhanced yield and/or nodulation of said leguminous plant or enhanced protein content of the seeds of said leguminous plant,
wherein the Bradyrhizobium japonicum strain has a deposit accession number LMG P-32018 or has at least 99.90% genomic sequence identity with a strain having the deposit accession number LMG P-32018, and wherein the Bradyrhizobium japonicum strain has retained plant growth and/or yield characteristics at temperatures between 10 and 25° C. of the deposited strain LMG P-32018.

27. The method of claim 23, wherein the Bradyrhizobium japonicum strain has at least 99.95% genomic sequence identity.

28. The method of claim 27, wherein the Bradyrhizobium japonicum strain has at least 99.98% genomic sequence identity.

29. The method of claim 23, wherein the Bradyrhizobium japonicum strain has at least one 16S rRNA according to SEQ ID No. 1.

30. The method of claim 23, wherein the Bradyrhizobium japonicum strain shows improved growth at 10° C. to 25° C. when grown on yeast mannitol agar (YMA) or in yeast mannitol broth (YMB).

31. The method of claim 23, wherein the Bradyrhizobium japonicum strain shows improved growth at 20 to 30° C. when grown in yeast mannitol broth (YMB), compared to Bradyrhizobium japonicum strain USDA6, strain G49, and/or strain 532C.

32. The method of claim 23, wherein the Bradyrhizobium japonicum strain induces red nitrogen-fixing nodules on roots of a plant.

33. The method of claim 23, the Bradyrhizobium japonicum strain induces at least 5% more nitrogen-fixing nodules on roots of a plant compared to Bradyrhizobium japonicum strain USDA6, or strain G49.

34. The method of claim 23, wherein the Bradyrhizobium japonicum strain increases a nitrogen status in a plant when inoculated with said Bradyrhizobium japonicum strain as measured via increased Soil Plant Analysis Development (SPAD) values, compared to said plant inoculated with Bradyrhizobium japonicum strain USDA6 or strain G49.

35. The method of claim 23, wherein the Bradyrhizobium japonicum strain increases leaf chlorophyll content a plant inoculated with said Bradyrhizobium japonicum strain compared to a seed of said plant inoculated with Bradyrhizobium japonicum strain USDA6.

36. The method of claim 23, wherein said leguminous plant is of the genus Glycine.

37. The plant seed according to claim 25, where the plant seed is a leguminous plant seed.

38. The plant seed according to claim 37, wherein the leguminous plant seed is a soybean seed.

39. The method of claim 25, wherein the Bradyrhizobium japonicum strain has at least one 16S rRNA according to SEQ ID No. 1.

40. The method of claim 26, wherein the Bradyrhizobium japonicum strain has at least one 16S rRNA according to SEQ ID No. 1.

41. The method of claim 25, wherein the Bradyrhizobium japonicum strain induces red nitrogen-fixing nodules on roots of said leguminous plant.

42. The method of claim 26, wherein the Bradyrhizobium japonicum strain induces red nitrogen-fixing nodules on roots of said leguminous plant.


SEQUENCE LISTING (1 sequence)

SEQ ID NO: 1 - RNA, 1068 nt, Bradyrhizobium japonicum
  source 1..1068: mol_type = rRNA
caaactcctacgggaggcagcagtggggaatattggacaatgggggcaaccctgatccagccatgccgcgtgagtgatgaaggccctagggttgtaaagctcttttgtgcgggaagataatgacggtaccgcaagaataagccccggctaacttcgtgccagcagccgcggtaatacgaagggggctagcgttgctcggaatcactgggcgtaaagggtgcgtaggcgggtctttaagtcaggggtgaaatcctggagctcaactccagaactgcctttgatactgaggatcttgagttcgggagaggtgagtggaactgcgagtgtagaggtgaaattcgtagatattcgcaagaacaccagtggcgaaggcggctcactggcccgatactgacgctgaggcacgaaagcgtggggagcaaacaggattagataccctggtagtccacgccgtaaacgatgaatgccagccgttagtgggtttactcactagtggcgcagctaacgctttaagcattccgcctggggagtacggtcgcaagattaaaactcaaaggaattgacgggggcccgcacaagcggtggagcatgtggtttaattcgacgcaacgcgcagaaccttaccagcccttgacatgtccaggaccggtcgcagagatgtgaccttctcttcggagcctggagcacaggtgctgcatggctgtcgtcagctcgtgtcgtgagatgttgggttaagtcccgcaacgagcgcaacccccgtccttagttgctaccatttagttgagcactctaaggagactgccggtgataagccgcgaggaaggtggggatgacgtcaagtcctcatggcccttacgggctgggctacacacgtgctacaatggcggtgacaatgggatgctaaggggcgacccttcgcaaatctcaaaaagccgtctcagttcggattgggctctgcaactcgagcccatgaagttggaatcgctagtaatcgtggatcagcacgccacggtgaatacgttcccgggccttgtacacaccgcccgtcacaccatgggagttggtttt